(19) United States
(12) Patent Application Publication
(10) Pub. No.: US 2006/0079739 A1
(43) Pub. Date: Apr. 13, 2006

(54) Multifunctional portable medical measurement device and display method thereof

(76) Inventors: Shiow-Chen Chen Wang, Chu-Nan (TW); Tsai-Yi Chen, Chu-Nan (TW); Tsong-Tseh Tsay, Chu-Nan (TW); Jih-Hsing Yeh, Chu-Nan (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/134,407

(22) Filed: May 23, 2005

(30) Foreign Application Priority Data
Oct. 12, 2004 (TW) 93130874

(51) Int. Cl. A61B 5/00

(52) U.S. Cl. 600/300

(57) ABSTRACT

A multifunctional portable medical measurement device and the display method thereof are proposed. The multifunctional portable medical measurement device has a first medical measurement unit, a second medical measurement unit, a key unit, a display unit, and a microprocessor. The microprocessor selects either a first measured value or a second measured value for output to the display unit according to the operation of the key unit. When the portable medical measurement device operates in the second measurement mode, a digital display for displaying only a numeral is used to display the second measured value of the second medical measurement unit. When the portable medical measurement device operates in the first measurement mode, a plurality of digital displays for displaying a numeral is combined to display the first measured value of the first medical measurement unit.

### BACKGROUND OF THE INVENTION

1. Field of the Invention

The present invention relates to a multifunctional portable measurement device and the display method thereof and, more particularly, to a portable measurement device capable of measuring medical.

2. Description of Related Art

Along with progress of the biochemical industry and development of the electronic industry, various medical measurement devices have been developed and improved upon. Multifunctional medical measurement devices emerge consequentially. For example, in the disclosure of “A system and method for monitoring medical treatment effect” of R.O.C. U.S. Pat. No. 516,948, a desktop medical measurement device 9 is proposed, as shown in FIG. 1. The measurements of blood sugar and blood pressure are integrated together. As shown in FIG. 2, a first microprocessor 70 is responsible for the operation of a blood pressure meter. The first processor 70 is connected with an operation key 71, a pressure sensor 72, a buzzer 73, an aeration pump 74, and a blow off valve 75. A second microprocessor 80 is responsible for the operation of a blood sugar meter. The second microprocessor 80 is connected with an LCD 81, an operation key 82, a memory 83, a realtime clock 84, a loudspeaker 85, and a blood sugar meter 86.

For the circuit shown in FIG. 2, although the measurements of blood sugar and blood pressure are integrated together, they are separately processed by individual microprocessors in practical operations. The measured result of blood pressure is sent to the second microprocessor 80 by the first microprocessor 70 to be displayed by the LCD 81. Although this kind of separate processing can lessen the operation burden to a single microprocessor, the cost increases. Moreover, the LCD 81 displays a numeral in a fixed way, whether the measured result is a blood pressure value or a blood sugar value. A patient with blood sugar problems usually has poor eyesight. Therefore, a common LCD 81 can't provide a conspicuous, large-size display because of limited product space to cause laborious reading for the patient.

### SUMMARY OF THE INVENTION

The primary object of the present invention is to provide a multifunctional portable medical measurement device and a display method thereof. A microprocessor is used to measure blood pressure and blood sugar. Moreover, the size of numeral displayed when measuring the blood sugar is larger than that displayed when measuring the blood pressure to facilitate reading for a patient with a blood sugar problem.

To achieve the above object, the present invention provides a multifunctional portable medical measurement device, which comprises a first medical measurement unit for output of a first measured value, a second medical measurement unit for output of a second measured value, a key unit, a display unit, and a microprocessor. The microprocessor controls actions of the first and second medical measurement units, and selects one of the first measured value and the second measured value for output to the display unit based on the operation of the key unit.

To achieve the above object, the present invention provides a multifunctional portable medical measurement device, which comprises a first shell and a second shell. The first shell comprises a first pivotal end, a first signal transmission terminal, a first medical measurement unit for output of a first measured value, a key unit, a display unit, and a microprocessor electrically connected to the first signal transmission terminal. The second shell comprises a second pivotal end, a second signal transmission terminal, and a second medical measurement unit. The second medical measurement unit is electrically connected to the first signal transmission terminal for output of a second measured value. The microprocessor controls actions of the first medical measurement unit and the second medical measurement unit, and selects one of the first measured value and the second measured value for output to the display unit based on operation of the key unit. The first pivotal end of the first shell can be movably combined with the second pivotal end of the second shell to let the first signal transmission terminal and the second signal transmission terminal contact each other so that the microprocessor can receive the second measured value.

To achieve the above object, the present invention also provides a display method for a medical measurement device. The medical measurement device comprises a first medical measurement unit and a second medical measurement unit. The display method comprises the steps of: using a digital display for displaying only a numeral to display a second measured value of the second medical measurement unit when the medical measurement device operates in the mode of the second medical measurement unit; switching the medical measurement device from the mode of the second medical measurement unit to the mode of the first medical measurement unit; and combining a plurality of digital displays for displaying only a numeral to display a first measured value of the first medical measurement unit when the medical measurement device operates in the mode of the first medical measurement unit.

### BRIEF DESCRIPTION OF THE DRAWINGS:

The various objects and advantages of the present invention will be more readily understood from the following detailed description when read in conjunction with the appended drawing, in which:

FIG. 1 is a diagram of a conventional desktop medical measurement device;

FIG. 2 is a circuit diagram of a conventional desktop medical measurement device;

FIG. 3 is a front view of a preferred embodiment of the present invention;

FIG. 4 is another perspective view of the preferred embodiment of the present invention;

FIG. 5 is yet another perspective view of the preferred embodiment of the present invention;

FIG. 6 is a circuit diagram of the preferred embodiment of the present invention;

FIG. 7 shows the display unit of the preferred embodiment of the present invention;

FIG. 8 shows a second display way according to the preferred embodiment of the present invention; and

FIG. 9 shows a first display way according to the preferred embodiment of the present invention.

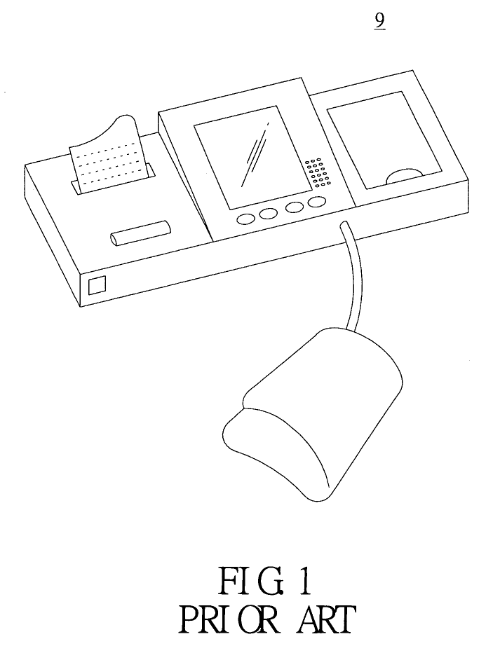

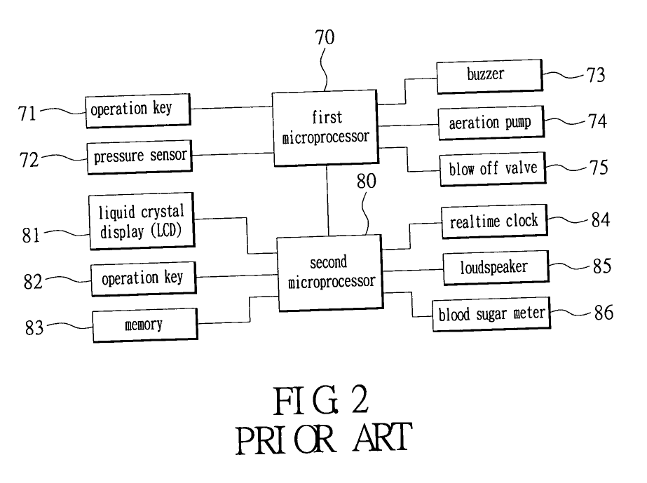

### DETAILED DESCRIPTION OF THE PREFERRED EMBODIMENTS

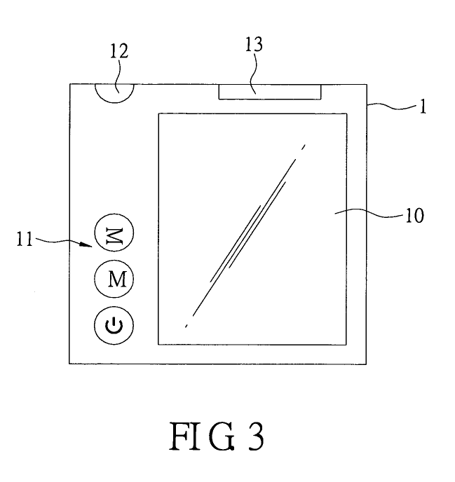

As shown in FIG. 3, the present invention provides a portable medical measurement device 1 having a first operation mode and a second operation mode. A key unit 11 is used to select the first or second operation as the present operation mode. A display unit 10 is used to display the measured result. The display unit 10 can be an LCD, a seven segment display, a dot-matrix display, or a vacuum tube display. In this embodiment, the first operation mode is for blood sugar measurement, while the second operation mode is for blood pressure measurement. However, the first and second operation modes are not limited to the measurement of blood sugar and blood pressure, but can be used for the medical measurement of ear temperature, body temperature, heart beat, pressure, cholesterol, uric acid, body fat, bio parameters, or heart tones.

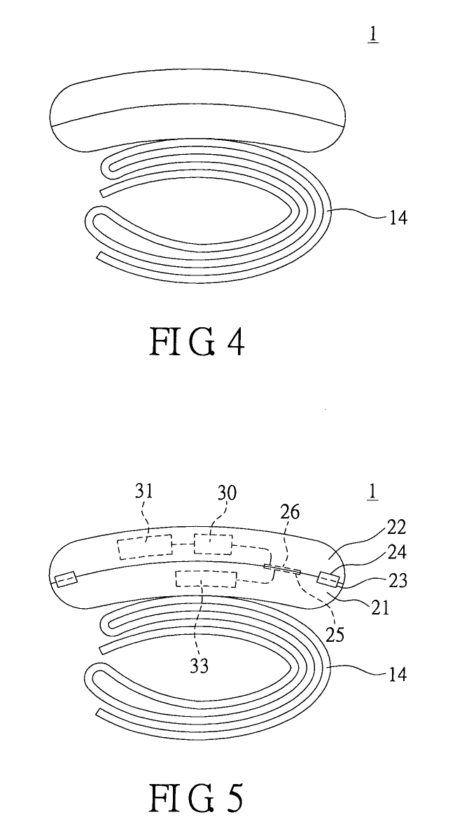

A strip slot 12 and a card reader 13 are disposed above the display unit 10 for use in the first operation mode. A ring belt buckle 14 is disposed at the back of the medical measurement device 1 for use in the second operation mode, as shown in FIG. 4. In the present invention, operation parts of the second operation mode of the medical measurement device 1 can further be designed to be detachable. As shown in FIG. 5, the medical measurement device 1 is composed of a first shell 22 and a second shell 21. The first shell 22 is responsible for the first operation mode, and comprises a microprocessor 30 and a first medical measurement unit 31. In this embodiment, the first medical measurement unit 31 can be an ear thermometer, a body thermometer, a heart beat sensor, a pressure sensor, a cholesterol sensor, a uric acid sensor, a body fat sensor, a biosensor, or a heart tones sensor. The second shell 21 is responsible for the second operation mode, and comprises a second medical measurement unit 33. In this embodiment, the second medical measurement unit 33 is a blood pressure sensor. Of course, the second medical measurement unit 33 can also be an ear thermometer, a body thermometer, a heart beat sensor, a pressure sensor, a cholesterol sensor, a uric acid sensor, a body fat sensor, a biosensor, or a heart tones sensor.

The first shell 22 and the second shell 21 can be movably combined together. The first pivotal end 24 of the first shell 22 and the second pivotal end 23 of the second shell 21 are connected together to let the first signal transmission terminal 26 of the first shell 22 and the second signal transmission terminal 25 of the second shell 21 contact each other so that the first shell 22 and the second shell 21 can transmit signals mutually via the first signal transmission terminal 26 and the second signal transmission terminal 25. The microprocessor 30 is electrically connected with the first signal transmission terminal 26. The second medical measurement unit 33 is electrically connected with the second signal transmission terminal 25. Both the first signal transmission terminal 26 and the second signal transmission terminal 25 have electric power transmission functions.

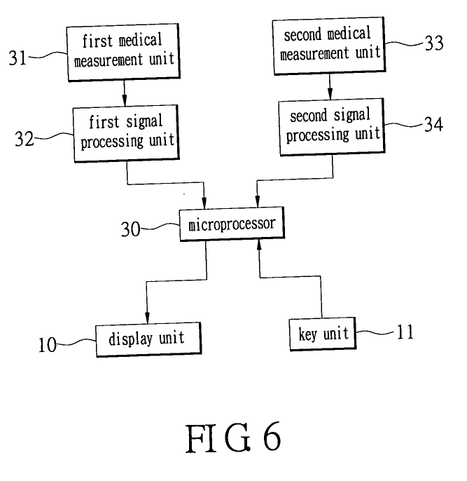

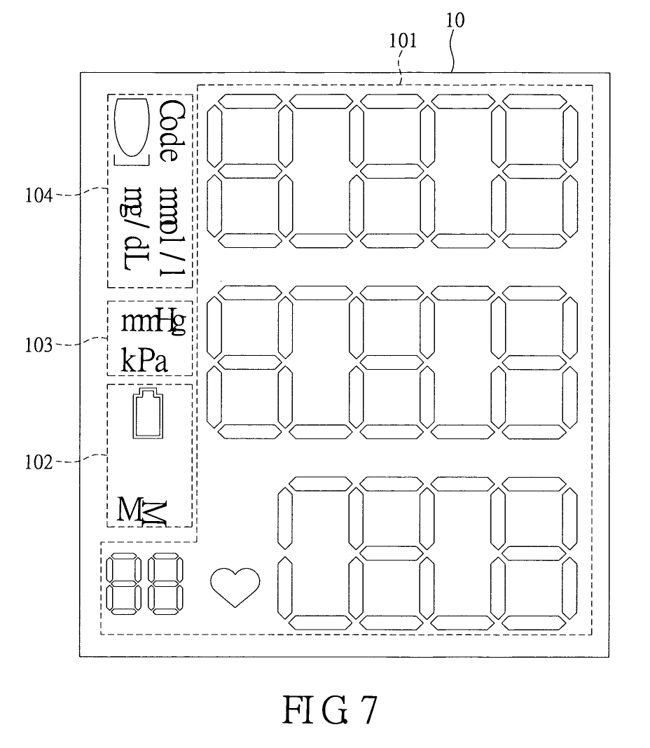

As shown in FIG. 6, the medical measurement device I comprises a first medical measurement unit 31, a second medical measurement unit 33, a first signal processing unit 32, a second signal processing unit 34, a microprocessor 30, a display unit 10, and a key unit 11. The first medical measurement unit 31 and the second medical measurement unit 33 jointly use the microprocessor 30. The microprocessor 30 controls actions of the first medical measurement unit 13 and the second medical measurement unit 33 based on the operation of the first medical measurement unit 31 or the key unit 11 to select either a first measured value output by the first medical measurement unit 31 or a second measured value output by the second medical measurement unit 33 for output to the display unit 10. The measured results of the first medical measurement unit 31 and the second medical measurement unit 33 are processed by the first signal processing unit 32 and the second signal processing unit 34, respectively, and are then sent to the microprocessor 30. The first signal processing unit 32 and the second signal processing unit 34 provide analog-to-digital conversion and signal amplification for the first measured value and the second measured value, respectively.

As shown in FIG. 7, the display unit 10 of the present invention has a numeral display area 101, a working state area 102, a second symbol area 103, and a first symbol area 104. The numeral display area 101 is used for displaying the first measured value or the second measured value, and is composed of a plurality of digital displays such as seven segment displays. The working state area 102 indicates the present operation mode (the first operation mode or the second operation mode) of the medical measurement device 1, and also provides a low battery alarm. The second symbol area 103 displays the unit of the second measured value. The first symbol area 104 displays the unit of the first measured value.

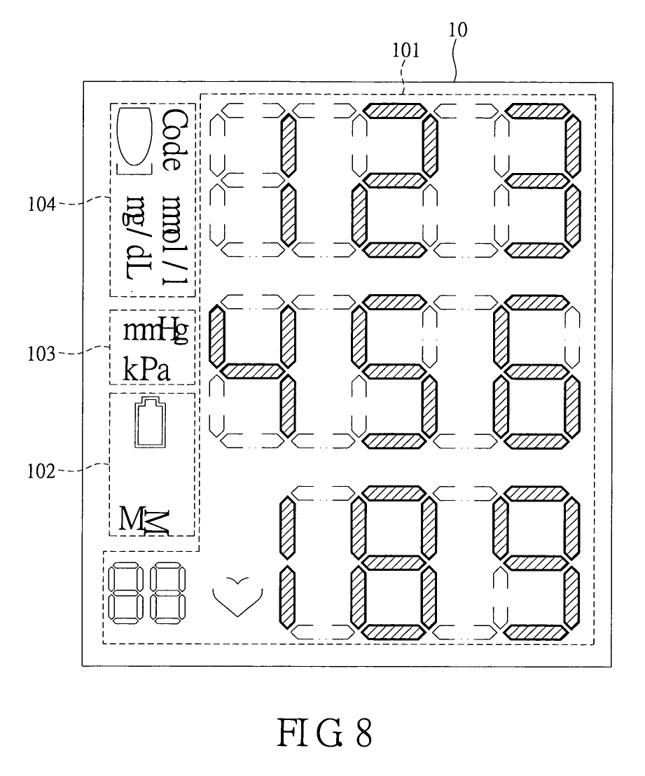

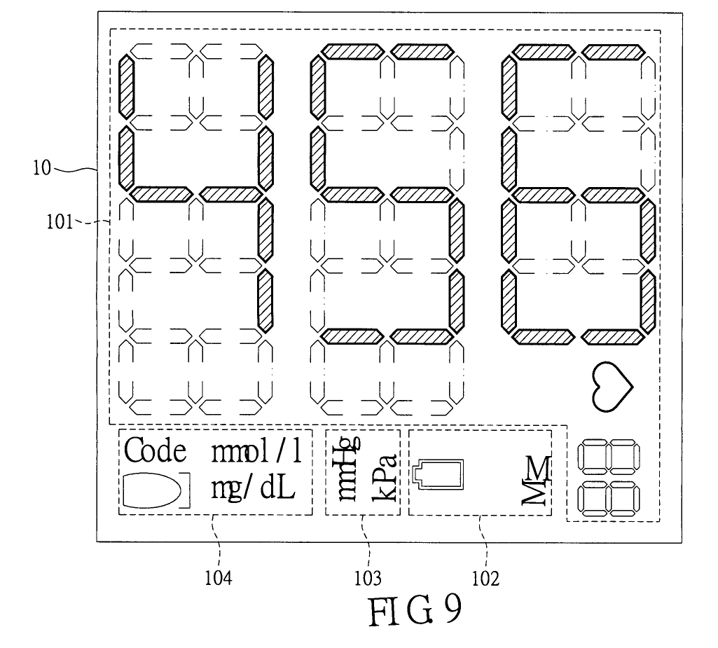

The display ways of the first measured value and the second measured value are different. FIG. 8 shows the second display way. The medical measurement device 1 operates in the second operation mode. Each digital display displays a digit for displaying the second measured value. When the key unit 11 operates the medical measurement device 1 in the first operation mode, the display way is shown in FIG. 9. Multiple digital displays are integrated for displaying a numeral. Moreover, the display direction is turned 90 degrees from that in FIG. 8. The displayed numeral in FIG. 9 is apparently larger that that in FIG. 8, hence facilitating reading for the patient. The display unit 10 shown in FIG. 8 is the same as that shown in FIG. 9; i.e., the display unit 10 can be jointly used.

Although the present invention has been described with reference to the preferred embodiment thereof, it will be understood that the invention is not limited to the details thereof. Various substitutions and modifications have been suggested in the foregoing description, and other will occur to those of ordinary skill in the art. Therefore, all such substitutions and modifications are intended to be embraced within the scope of the invention as defined in the appended claims.

I claim:

1. A multifunctional portable medical measurement device at least comprising:
a first medical measurement unit for output of a first measured value;
a second medical measurement unit for output of a second measured value;
a key unit;
a display unit; and
a microprocessor for controlling actions of said first medical measurement unit and said second medical measurement unit and selecting one of said first measured value and said second measured value for output to said display unit.

2. The multifunctional portable medical measurement device as claimed in claim 1, wherein said first medical measurement unit and said second medical measurement unit are a blood sugar sensor, a blood pressure sensor, an ear temperature sensor, a body temperature sensor, a heart beat sensor, a pressure sensor, a cholesterol sensor, a uric acid sensor, a body fat sensor, a biosensor, or a heart sounds sensor, respectively.

3. The multifunctional portable medical measurement device as claimed in claim 1, wherein said display unit is an LCD, a seven segment display, a matrix display, or a vacuum tube display.

4. The multifunctional portable medical measurement device as claimed in claim 1, wherein said display unit has a numeral display area, a working state area, a second symbol area, and a first symbol area.

5. The multifunctional portable medical measurement device as claimed in claim 4, wherein said numeral display area comprises a plurality of digital displays.

6. The multifunctional portable medical measurement device as claimed in claim 5, wherein said digital displays are seven segment displays.

7. The multifunctional portable medical measurement device as claimed in claim 1, further comprising a first signal processing unit, wherein the first signal processing unit is used to provide analog-to-digital conversion and signal amplification for said first measured value.

8. The multifunctional portable medical measurement device as claimed in claim 1, further comprising a second signal processing unit, wherein the second signal processing unit is used to provide analog-to-digital conversion and signal amplification for said second measured value.

9. A multifunctional portable medical measurement device at least comprising:
a first shell comprising:
a first pivotal end;
a first signal transmission terminal;
a first medical measurement unit for output of a first measured value;
a key unit;
a display unit; and
a microprocessor electrically connected to said first signal transmission terminal; and
a second shell comprising a second pivotal end, a second signal transmission terminal, and a second medical measurement unit, said second medical measurement unit being electrically connected to said first signal transmission terminal for output of a second measured value;
whereby said microprocessor controls actions of said first medical measurement unit and said second medical measurement unit and selects one of said first measured value and said second measured value based on the operation of said key unit for output to said display unit, and said first pivotal end of said first shell is movably combined with said second pivotal end of said second shell to let said first signal transmission terminal and said second signal transmission terminal contact each other so that said microprocessor can receive said second measured value.

10. The multifunctional portable medical measurement device as claimed in claim 9, wherein said first medical measurement unit and said second medical measurement unit are a blood sugar sensor, a blood pressure sensor, an ear temperature sensor, a body temperature sensor, a heart beat sensor, a pressure sensor, a cholesterol sensor, a uric acid sensor, a body fat sensor, a biosensor, or a heart sounds sensor, respectively.

11. The multifunctional portable medical measurement device as claimed in claim 9, wherein said display unit is an LCD, a seven segment display, a matrix display, or a vacuum tube display.

12. The multifunctional portable medical measurement device as claimed in claim 9, wherein said display unit has a numeral display area, a working state area, a second symbol area, and a first symbol area.

13. The multifunctional portable medical measurement device as claimed in claim 12, wherein said numeral display area comprises a plurality of digital displays.

14. The multifunctional portable medical measurement device as claimed in claim 13, wherein said digital displays are seven segment displays.

15. The multifunctional portable medical measurement device as claimed in claim 9 further comprising a first signal processing unit, wherein the first signal processing unit is used to provide analog-to-digital conversion and signal amplification for said first measured value.

16. The multifunctional portable medical measurement device as claimed in claim 9 further comprising a second signal processing unit, wherein the second signal processing unit is used to provide analog-to-digital conversion and signal amplification for said second measured value.

17. A display method of a portable medical measurement device, said portable medical measurement device comprising a first medical measurement unit and a second medical measurement unit, said display method comprising the steps of:
using a digital display for displaying only a numeral to display a second measured value of said second medical measurement unit when said portable medical measurement device operates in a mode of said second medical measurement unit;
switching said portable medical measurement device form the mode of said second medical measurement unit to a mode of said first medical measurement unit; and
combining a plurality of digital displays for displaying only a numeral to display a first measured value of said first medical measurement unit when said portable medical measurement device operates in the mode of said first medical measurement unit.

18. The display method of a portable medical measurement device as claimed in claim 17, wherein a display direction of said portable medical measurement device when operating in the mode of said first medical measurement unit differs by an angle from a display direction when operating in the mode of said second medical measurement unit.

19. The display method of a medical measurement device as claimed in claim 18, wherein said angle is 90 degrees.
